(19) United States
(12) Patent Application Publication
(10) Pub. No.: US 2017/0086704 A1
(43) Pub. Date: Mar. 30, 2017

(54) ELECTRONIC SKIN

(71) Applicant: INTEL CORPORATION, Santa Clara, CA (US)

(72) Inventors: Paul J. Gwin, Orangevale, CA (US); Mark E. Sprenger, Folsom, CA (US)

(73) Assignee: INTEL CORPORATION, Santa Clara, CA (US)

(21) Appl. No.: 14/866,807

(22) Filed: Sep. 25, 2015

(51) Int. Cl. A61B 5/053; A61B 5/103; A61B 5/11; A61B 5/00

(52) U.S. Cl. CPC A61B 5/0531; A61B 5/6807; A61B 5/1038; A61B 5/742; A61B 5/7475; A61B 5/0015; A61B 5/112; A61B 2562/0247

(57) ABSTRACT

An electronic skin is described herein. The electronic skin comprises a flexible capacitive sensor to capture a change in capacitance, wherein the change in capacitance is to translate to sheer, tensile, and torsion values. The electronic skin also comprises at least one section, wherein the one section is dielectrically separated from another section and the sheer, tensile, compression and torsion values are continuous across the at least one section and the another section.

### TECHNICAL FIELD

The present techniques relate to a sensor. In particular, the present techniques relates to the sensing capability of human flesh.

### BACKGROUND

Wearable computing devices incorporate a number of methods for interacting with humans and tracking various parameters. For example, wearable fitness devices can track how far a person has walked, flights of stairs climbed, heart rate, etc. Examples of sensors used in wearable devices include resistive sensors and capacitive sensors, among others.

### BRIEF DESCRIPTION OF THE DRAWINGS

Certain exemplary embodiments are described in the following detailed description and in reference to the drawings, in which:

FIG. 1 is a block diagram of a computing device that can be used with an electronic skin;

FIG. 2A is an illustration of a flexible capacitive sensor;

FIG. 2B is an illustration of an electronic skin with insulators;

FIGS. 3A-3D are illustrations deformation of the flexible capacitive sensor, in accordance with an embodiment;

FIG. 4A is an illustration of an electronic skin sole insert;

FIG. 4B is a cross-section of a shoe with the electronic skin as the sole;

FIG. 5 is a process flow diagram of an example of a method of manufacturing an electronic skin; and

FIG. 6 is a process flow diagram of an example of a method of using an electronic skin.

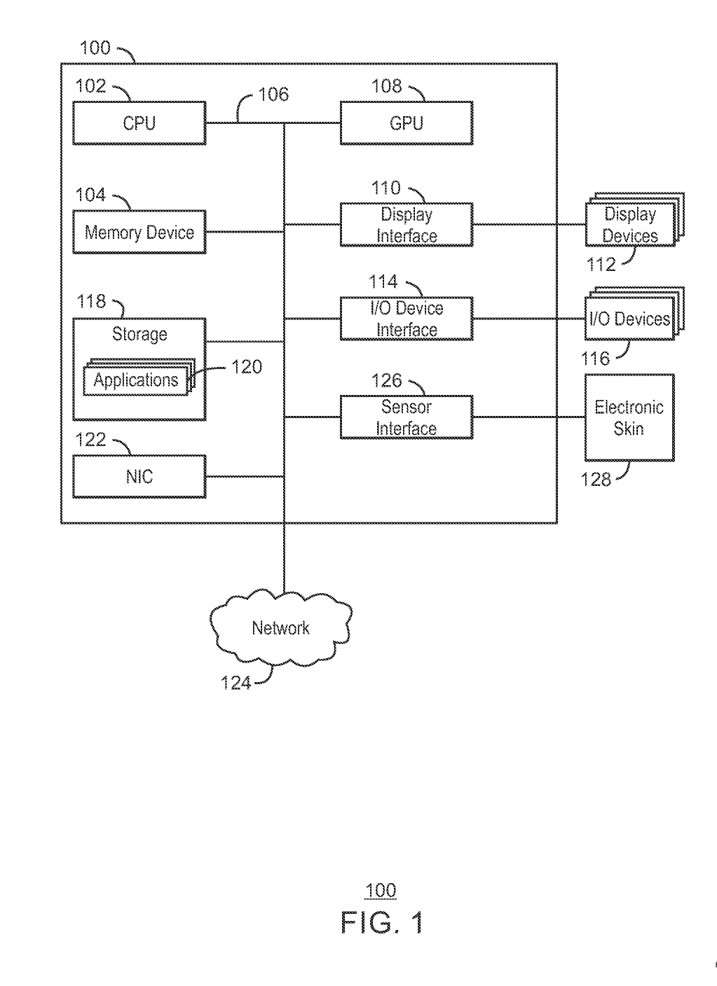

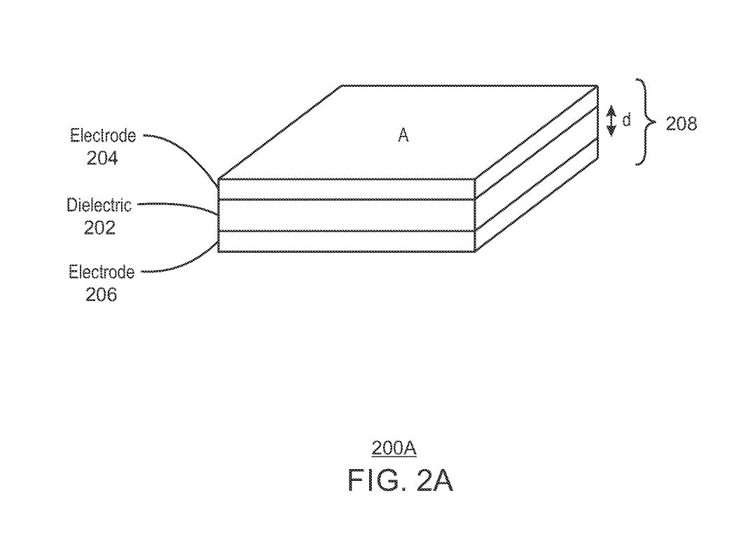

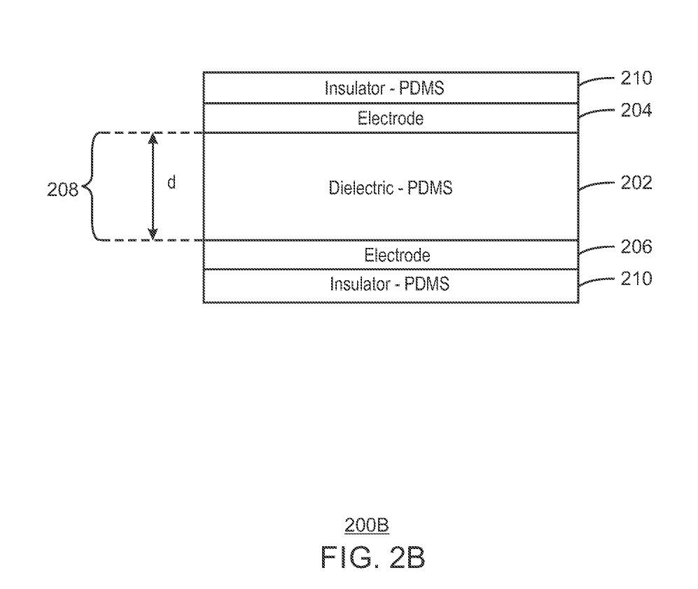

The same numbers are used throughout the disclosure and the figures to reference like components and features. Numbers in the 100 series refer to features originally found in FIG. 1; numbers in the 200 series refer to features originally found in FIG. 2; and so on.

### DETAILED DESCRIPTION OF SPECIFIC EMBODIMENTS

Current methods of pressure and motion analysis involve static pressure measurements from the simplest foot print creation (ink), and complex slow motion video analysis using sophisticated cameras and treadmills/sensor platforms. In some cases pressure sensors are used. The pressure sensor may have multiple sensing circuits that are based on decreasing electrical resistance as pressure is applied to the electrodes. Capacitance methods may be used, but the capacitance methods are used with axial movement cells. Axial movement cells with capacitance methods sense a unidirectional force that only captures a component of the force vector while the fidelity of the resultant direction and magnitude vector are unknown.

Embodiments disclosed herein provide techniques for an electronic skin. In particular, embodiments disclosed herein provide techniques for a human like wearable sensing technology. As opposed to rigid mechanical sensing, the present techniques enable flexible sensing with greater degrees of freedom with the ability to capture three dimensional complex motion.

FIG. 1 is a block diagram of a computing device 100 that can be used with an electronic skin. The computing device 100 can be, for example, a laptop computer, desktop computer, tablet computer, mobile device, or server, among others. In particular, the computing device 100 can be a mobile device such as a cellular phone, a smartphone, a personal digital assistant (PDA), phablet, or a tablet. The computing device 100 can include a central processing unit (CPU) 102 that is configured to execute stored instructions, as well as a memory device 104 that stores instructions that are executable by the CPU 102. The CPU can be coupled to the memory device 104 by a bus 106. Additionally, the CPU 102 can be a single core processor, a multi-core processor, a computing cluster, or any number of other configurations. Furthermore, the computing device 100 can include more than one CPU 102. The memory device 104 can include random access memory (RAM), read only memory (ROM), flash memory, or any other suitable memory systems. For example, the memory device 104 can include dynamic random access memory (DRAM).

The computing device 100 can also include a graphics processing unit (GPU) 108. As shown, the CPU 102 can be coupled through the bus 106 to the GPU 108. The GPU 108 can be configured to perform any number of graphics operations within the computing device 100. For example, the GPU 108 can be configured to render or manipulate graphics images, graphics frames, videos, or the like, to be displayed to a user of the computing device 100. In some embodiments, the GPU 108 includes a number of graphics engines, wherein each graphics engine is configured to perform specific graphics tasks, or to execute specific types of workloads.

The CPU 102 can be linked through the bus 106 to a display interface 110 configured to connect the computing device 100 to a display device 112. The display device 112 can include a display screen that is a built-in component of the computing device 100. The display device 112 can also include a computer monitor, television, or projector, among others, that is externally connected to the computing device 100.

The CPU 102 can also be connected through the bus 106 to an input/output (I/O) device interface 114 configured to connect the computing device 100 to one or more I/O devices 116. The I/O devices 116 can include, for example, a keyboard and a pointing device, wherein the pointing device can include a touchpad or a touchscreen, among others. The I/O devices 116 can be built-in components of the computing device 100, or can be devices that are externally connected to the computing device 100.

The computing device also includes a storage device 118. The storage device 118 is a physical memory such as a hard drive, a solid state drive, an optical drive, a thumbdrive, an array of drives, or any combinations thereof. The storage device 118 can also include remote storage drives such as used for cloud computing applications. The storage device 118 includes any number of applications 120 that are configured to run on the computing device 100.

The computing device 100 can also include a network interface controller (NIC) 122. The NIC 122 can be configured to connect the computing device 100 through the bus 106 to a network 124. The network 124 can be a wide area network (WAN), local area network (LAN), or the Internet, among others.

The computing device 100 also includes a flexible capacitive sensor interface 126 to connect the computing device 100 to an electronic skin 128. The electronic skin 128 may include a flexible, capacitive flexible capacitive sensor. The capacitance of the flexible capacitive sensor 128 is changed by deforming the flexible capacitive sensor 128. In some cases, the electronic skin 128 includes electrodes layered with insulators. For example, the insulator can be a silicone material, such as polydimethylsiloxane (PDMS).

The deformation of the electronic skin may occur by contorting the electronic skin in some form. For example, the electronic skin may experience any sort of deformation, such as twist, bend, stretch, compression, etc. The deformation results in a change of capacitance at various points on the electronic skin. Pressures throughout the electronic skin may be determined based on the change in capacitance. Additionally, motion may be inferred by the change in capacitance by using an algorithm that resolves the dynamic capacitance change of the electronic skin into motion analysis. Moreover, the change in capacitance across the electronic skin can be used to determine the motion of the object to which the electronic skin is attached, in terms of a roll, pitch or yaw. Note that the change in capacitance is measured for each cell or section of the electronic skin as described below. The adjacent cell changes can be compared and correlated with specific motions, such as a twisting motion. In embodiments, this calculation is performed in a sensor algorithm, and the calibration of the electronic skin can include various deformations that would be realized in pitch/yaw/roll situations.

The block diagram of FIG. 1 is not intended to indicate that the computing device 100 is to include all of the components shown in FIG. 1. Further, the computing device 100 can include any number of additional components not shown in FIG. 1, depending on the details of the specific implementation.

The electronic skin enables capacitive sensing in a fashion that enables a large degree of freedom in order to replicate the human flesh sensing capability. For ease of description, a shoe may be used to describe the present techniques. However, a shoe is only an exemplary embodiment, and the present techniques can be used alone and with other wearable devices. For example, the electronic skin may be applied to the back of a human to continually sense back positioning for detecting of back problems and poor posture. In embodiments, the electronic skin may be applied directly to the back via an adhesive or tape. The electronic skin may also be applied to the back via a shirt or other article of clothing that holds the electronic skin in place against the back. Additionally, the electronic skin may be applied to knees, legs, ankles and thighs to continually track a person's gait, movement of the knee, etc. The electronic skin can be applied to the knees, legs, ankles and thighs via an adhesive or tape, and can also be held in place using pants, wraps, or other articles of clothing that can cover the knees, legs, ankles or thighs. Similarly, the electronic skin may be applied to the shoulders, arms, and hands to detect the onset of motor skill deficiencies via tape, adhesives, gloves, wraps, or other articles of clothing that can cover the arms and hands. In embodiments, the posture and/or position of a subject over time can be logged to track/detect degenerating back or knee issues. Moreover, tracking the posture and/or position of a subject over time enables the rehabilitation of injuries to various areas of the subject to be monitored.

While particular uses have been described, it should be understood that these uses are exemplary, and the present techniques can be used in any application that can track parameters of a subject. As used herein, subject parameters are measurable characteristics of a subject that can be determined based on pressure and motion associated with the subject. While a shoe sensing application is used to demonstrate the present techniques, the present techniques are widely applicable to multiple wearable sensing applications and should not be confused with a single shoe sensing invention.

The electronic skin enables strain energy to be tracked via a change in capacitance as loads are applied and/or transferred across the electronic skin. The strain energy imparted to the electronic skin causes a change in shape, and the resulting change in capacitance is interpreted as a load necessary to cause a magnitude of capacitance change. The shape change from any stress or change at the sensor causes a capacitance change, which occurs by deformation along the x, y, and z axes and deformation due to rotation. This capacitance change is sent to the wearable device for processing, or storage and post processing. The strain energy may be interpreted as pressure, pressure distribution, force, force vectors, flexure, and shear stress, depending on how the sensor is segmented and arranged. The electronic skin also enables a sensing capability that is based on the strain energy applied to the sensor allowing capture of a signature based on the deformation of the sensor rather than only pressure. Sensing methods described herein are able to determine deformation and capacitance change from the principal stresses and then combine those stresses to get an effective stress. The effective stress results in a signature, where the signature is a capacitance change with unique inputs.

FIG. 2A is an illustration of a flexible capacitive sensor 200A. The flexible capacitive sensor 200A includes a dielectric material 202 layered between electrodes 204, 206. While the flexible capacitive sensor 200A is illustrated as a single dielectric 202 layered between two electrodes 204, 206, it is to be understood that the flexible capacitive sensor 200A can include additional dielectric and electrode layers, depending on the design of the flexible capacitive sensor 200A. In an example, electrode 204 can be the same material as electrode 206. In another example, electrode 204 can be a different material from electrode 206. The dielectric 202 and the electrodes 204, 206 can be formed of a polymer, such as a flexible polymer. The polymer may also be an amorphous polymer. In examples, the polymer can be a silicone, such as polydimethylsiloxane (PDMS). Furthermore, the electrodes 204, 206 can be a silicone and a conducting medium, such as carbon, or any other suitable conducting material, compounded into the silicone. This results in highly flexible electrodes.

The high flexibility of the flexible capacitive sensor 200A enables the flexible capacitive sensor 200A to be highly conformable compared to other sensors that track motion and pressure. Accordingly, the flexible capacitive sensor 200A can be applied to a surface with a variety of shapes, including flat surfaces and curved surfaces. In the process of tracking the movement and pressure resulting from a person, forces may be applied to the flexible capacitive sensor 200A and, regions of the flexible capacitive sensor 200A may deform more than other regions of the flexible capacitive sensor 200A. This results in a change in the capacitance of these deformed regions when compared to the less or not deformed regions of the flexible capacitive sensor 200A.

By calibrating the flexible capacitive sensor 200A after forming the flexible capacitive sensor 200A to the curved surface, the change in capacitance can be further attributed to pressures from a subject. Calibration of the flexible capacitive sensor creates a baseline or expected set of deformations across the electronic skin. Further deformations as a subject moves can be compared to this baseline. The flexible capacitive sensor 200A additionally supports a strain of 400% and higher. In embodiments, the strain may be up to 400%. This high supported strain enables the force/deflection curve of the flexible capacitive sensor 200A to endure long deformations without damage. Moreover, the high strain means the flexible capacitive sensor can extend the range of sensing to a wide range of motions that induce high deformations to the material. For example, the flexible capacitive sensor can be applied to an elbow and track bending through a complete range of motion and sensing. In this manner, the flexible capacitive sensor can transform with a lower force exertion to deform the sensor compared to previous solutions.

The capacitance of the flexible capacitive sensor 200A is changed by deforming the flexible capacitive sensor 200A. In some cases, deforming the flexible capacitive sensor means applying pressure to the flexible capacitive sensor such that the shape of the flexible capacitive sensor is altered. Capacitance is a function of the electrode area A, the electrode charge, the distance d between electrodes, and the permittivity of the volume between charge plates. When a force is exerted on the flexible capacitive sensor 200A, the electrode area A deforms and the distance d changes, which in turn changes the capacitance of the flexible capacitive sensor 200A. The capacitance is sensed by a circuit (not illustrated) and correlated to a force applied to the flexible capacitive sensor 200A.

The force applied to the flexible capacitive sensor 200A and the resulting shape change of the flexible capacitive sensor 200A may be used to determine changes of motion from the subject, or changes in the type of loading on the flexible capacitive sensor 200A from the subject. In particular, a force of the same or different magnitude can be applied in various directions at the flexible capacitive sensor 200A, and the magnitude of change in the capacitance of the flexible capacitive sensor 200A will vary based on the type of loading. In embodiments, a control algorithm can detect a variation in capacitance of neighboring regions and determine the direction of the force.

The illustration of FIG. 2A is not intended to indicate that the flexible capacitive sensor 200A is to include all of the components shown in FIG. 2A. Further, the flexible capacitive sensor 200A can include any number of additional components not shown in FIG. 2A, depending on the details of the specific implementation.

FIG. 2B is an illustration of an electronic skin 200B with insulators 210. The electronic skin 200B can be a multi-electronic skin, which detects multiple points of contact. An outer insulator 210 (the insulator contacted by a user) can be a more rigid structure when compared to the flexible capacitive sensor within the electronic skin 200B that moderates the shape factor imparting a load on the flexible capacitive sensor. In embodiments, the insulators 210 completely surround the flexible capacitive sensor. The insulators 210 may be formed from a silicone material, such as polydimethylsiloxane (PDMS).

In embodiments, materials used for the electronic skin can be designed for the intended purpose. For example, a stiffer material may be used in a shoe where high loading will occur. The material would be able to withstand the forces and at the same time not completely saturate the sensor capability. Accordingly, when a stiffer material is used, the stiffer material requires more force to deform the stiffer material. In some cases, the sensor including a stiffer material may not have as large of a capacitance change when compared to less stiff materials. Thus, in some embodiments, changing the stiffness of the material used for the sensor may be referred to as capacitance change moderation. If the electronic skin is intended for wearing on the back, capacitance change moderation may result in a softer material used to conform to the back more easily. Accordingly, the PDMS of an electronic skin may be formulated to be relatively stiffer or less stiff, depending on the capacitance change moderation and intended use of the electronic skin.

The electronic skin 200B can be applied to any surface on a subject. For example, the electronic skin 200B can be applied to any surface of the user's body where tracking is desired. The electronic skin 200B may also be applied to any surface with which a user interacts. For example, the electronic skin can enable a two-dimensional multi-touch capability. In such an implementation, harder, more forceful touches along with varying translations can be obtained from the electronic skin.

The type of loading (direction and shape deformation characteristics) can be calibrated, patterned, and sensed for intelligent interpretation of the force signature. This can yield greater usability and even some level of security. For example, in a shoe application, the electronic skin 200B may include insulators 210 that are to protect the sensor from damage by a foot. The insulators 210 may also protect the electronic skin from excess humidity, bacteria, and odors that may be generated by a foot. When a user is wearing the shoe, a baseline pressure profile can be obtained by first calibrating the electronic skin 200B to negate the initial change in capacitance that occurs when the electronic skin 200B is deformed from its original shape, prior to a load on the electronic skin. In examples, this baseline pressure profile may occur when the user is standing and applying pressure to the electronic skin 200B, or when the user is not applying pressure to the electronic skin 200B. The type of baseline pressure profile obtained depends on the particular use of the electronic skin 200B.

Accordingly, calibrating the electronic skin includes normalizing the sensor to a particular loading condition to create baseline or ground profile. In some use cases, calibrating may also be necessary depending on if clothing is worn over the sensor. Calibrating, in the case of clothing, incorporates the effects of the garment and resets a baseline with the garment included. Calibrating with a garment in place potentially mitigates or makes acceptable the effects of the garment. Calibrating prevents the use of shirts, gloves, socks, and other wearables that could be placed between the electronic skin and the source of pressure, or on top of the electronic skin and the source of pressure.

In embodiments, the baseline profile is to include shear, tensile, torsion, and compressive values. The shear stress on the electronic skin measures the stress that is coplanar with a cross section of the electronic skin. The cross sections may occur in an x, y, and z plane, resulting in three degrees of freedom with respect to the shear stress. Tensile stress results from stretching, and capacitance occurs as a result of this stretching. The tensile stress may occur in an x, y, and z direction, resulting in three degrees of freedom with respect to the tensile stress. Compressive stress results from compression, and a capacitance change occurs as a result of this compression. Additionally, the torsion load on the electronic skin causes torsional stress, which results in another capacitance change. The twisting may occur along x, y, and z axes, resulting in three degrees of freedom with respect to the torsion values. In total, the electronic skin may measure values associated with pressure and motion using nine degrees of freedom. Further, the twisting along three axes enables deformation to be measured as a function of capacitance change, thereby enabling analysis of the motion or change of shape of the object according to the capacitance change. Moreover, any other types of deformation, such as shear stress, bending, compressive, tensile, etc. can occur along the three axes and then be analyzed to determine the motion or change of shape of the object according to the capacitance change. While one particular type force may be used to describe the present techniques, any type force or load may be used to electronically interpret the motion or pressures imparted to the subject wearing the sensor.

The present techniques may also detect electrode resistivity change. The resistivity of the electrodes within the electronic skin may change or drift as a function of cycle count or fatigue, where the cycle count refers to the number of times the electronic skin has been cycled through various uses. The resistivity of the electrodes within the electronic skin may change or drift as a result of water content or temperature change. Accordingly, the baseline profile of the electronic skin may include measuring the resistance to enable real-time calibration of the sensor as changes occur in use conditions. The resistivity is sensed simultaneously with the dynamic capacitance change while capacitor element is physically distorted. Because the resistivity will change corresponding to the type of deformation, knowing the resistivity can be used to determine if the capacitance changed is due to compressive or tensile deformation of the sensor, which is key in motion and force analytics and results in a “smarter” sensor.

The change in capacitance of the electronic skin 200B initiates a response in a computing device coupled with the electronic skin 200B. This change in capacitance, along with the resulting shear, tensile, and torsion values can be used to analyze a number of subject parameters. For example, in the case of an electronic skin being applied to a show, gait analysis and foot function may be observed. Force may be plotted versus time, and pressure profiles of the foot may be obtained.

Because force is an analog input, as the amount of force changes, the response of the computing device can also change. In an example, the computing device can be calibrated to initiate different responses depending on the amount of force. These responses can be calibrated to respond linearly or nonlinearly to the force. For example, when a small force is applied to the electronic skin 200B, a first response can be initiated. When a large force is applied to the electronic skin 200B, a second response can be initiated. In another example, the electronic skin 200B can be calibrated to a particular user. For example, a first user can calibrate a first range of force to apply to the electronic skin 200B and a second user can calibrate a second range of force to apply to the flexible capacitive sensor 200. When a force within the first range of force is applied to the flexible capacitive sensor 200, the computing device can initiate the first user's profile. When a force within the second range of force is applied to the flexible capacitive sensor 200, the computing device can initiate the second user's profile. In this manner, the electronic skin can be used on a plurality of subjects by gyms, medical facilities, gaming facilities, and the like. Further, user profiles can be defined by a patterned signature. A patterned signature, as used herein, refers to a type of loading that occurs in a pattern. This pattern can be recognized by a computing device and matched with a particular user profile.

The electronic skin 200B can support peripheral device applications. For example, the electronic skin 200B can be a device that is removably coupled to a computing device. Moreover, in examples, the electronic skin 200B can be shaped as a large rubber band that extends around a subject, or other geometries. The electronic skin 200B can communicate wirelessly with the computing device as the electronic skin 200B is manipulated to initiate a response from the computing device. For example, the electronic skin 200B can act as a sleeve along the arm of a human subject. The changes in the capacitance from the electronic skin can be broken down into contributions from the primary deformation inputs to determine the input that caused the deformation. A primary deformation input is a force vector input along the principal axis or component of a resultant force vector that is superimposed onto the principal axis (X, Y, Z). The primary deformation input includes component vectors that are sensed by the capacitive sensor and can be used to calculate the resultant vector which is the actual input force direction and magnitude. As a result, while the change in capacitance is a magnitude and may not include a direction, by analysis the magnitude and direction of force on the principal axis can be determined based on the total capacitance change.

The electronic skin 200B can be placed in any wearable device configured to track pressure, motion, and other stresses. For example, the electronic skin may be used as the sole of a shoe. The electronic skin may also be placed in a shirt, in a pair of pants, etc., as described above. The electronic skin 200B can include any suitable number of layers 202, 204, 206, and 210, depending on the design of the electronic skin 200B. In embodiments, the electronic skin is less than 500 μm thick.

The electronic skin 200B sensor may be a composite construction of a thin elastomer such as silicone, an electrode such as exfoliated graphite compounded with silicone, and a dielectric insulator such as silicone, mylar, etc. The arrangement of these materials creates a capacitor with an inner dielectric (silicone), a charge plate on each side of the dielectric (graphite silicone), and an insulator on both outer sides (silicone). In embodiments, the electronic skin can be manufactured from 0.2 to several millimeters thickness (no real upper limit).

Additionally, the electronic skins 400 to be created at low cost. The electronic skin 200B can be less than 500 μm thick, such as less than 200 μm thick, whereas typical axial movement cells are not less than 2.8 mm thick. For example, each layer 202, 204, 206, and 210 can be 30 μm thick, resulting in an electronic skin 120 μm thick. Further, the electronic skin 200B can have a supportable strain limited only by the materials of the electronic skin 200B. For example, the electronic skin 200B can have a strain capability up to 800% or more, such as up to 700%, up to 600%, up to 500%, up to 400, or up to 300%. For example, the electronic skin 200B can have a strain capability of 350%. By contrast, the typical electronic skin can only support a strain up to 2%. This limited supportable strain of the typical electronic skin limits potential applications of the typical electronic skin.

The illustration of FIG. 2B is not intended to indicate that the electronic skin 200B is to include all of the components shown in FIG. 2. Further, the electronic skin 200B can include any number of additional components not shown in FIG. 2, depending on the details of the specific implementation.

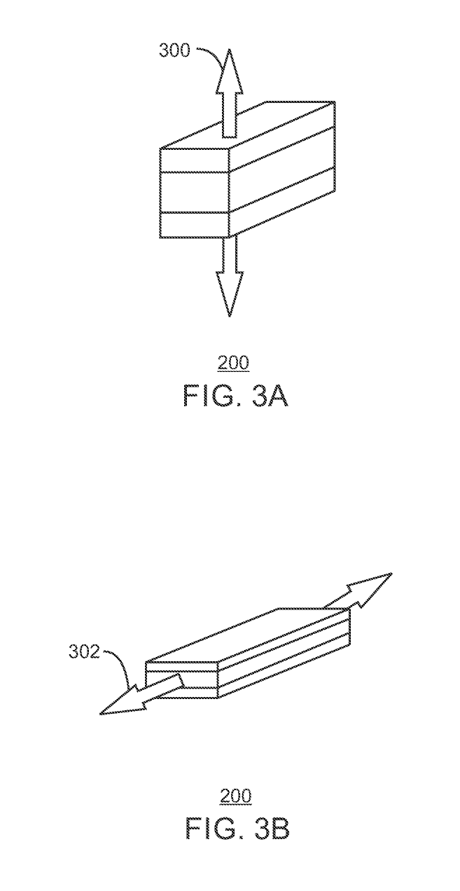

FIGS. 3A-3D are illustrations of deformation of an electronic skin 200A or 200B. The capacitance of the electronic skin 200A or 200B can be changed by deforming the electronic skin 200A or 200B. The electronic skin 200A or 200B can be deformed in any number of ways. For example, as illustrated by FIG. 3A, the electronic skin 200A or 200B can be deformed by stretching the skin vertically 300. In another example, illustrated by FIG. 3B, the electronic skin 200A or 200B can be deformed by stretching the skin horizontally 302. In a further example, illustrated by FIG. 3C, the electronic skin 200A or 200B can be deformed by compressing the electronic skin 200A or 200B vertically 304. In other examples, illustrated by FIG. 3D, the electronic skin 200A or 200B can be bent 306, inducing strain in the electronic skin 200, or twisted. In addition, the electronic skin 200A or 200B can be deformed in any other way not illustrated here.

The electronic skin 200A or 200B can be designed to react to any deformation. For example, the electronic skin 200A or 200B can be designed to react to a light touch on the electronic skin 200A or 200B resulting in a small deformation. In another example, the electronic skin 200A or 200B can be designed to react to a heavy touch on the electronic skin 200A or 200B resulting in a large deformation or a small deformation. In another example, the electronic skin 200A or 200B can measure the degree of deformation of the electronic skin 200A or 200B and can initiate a response based on the degree of deformation.

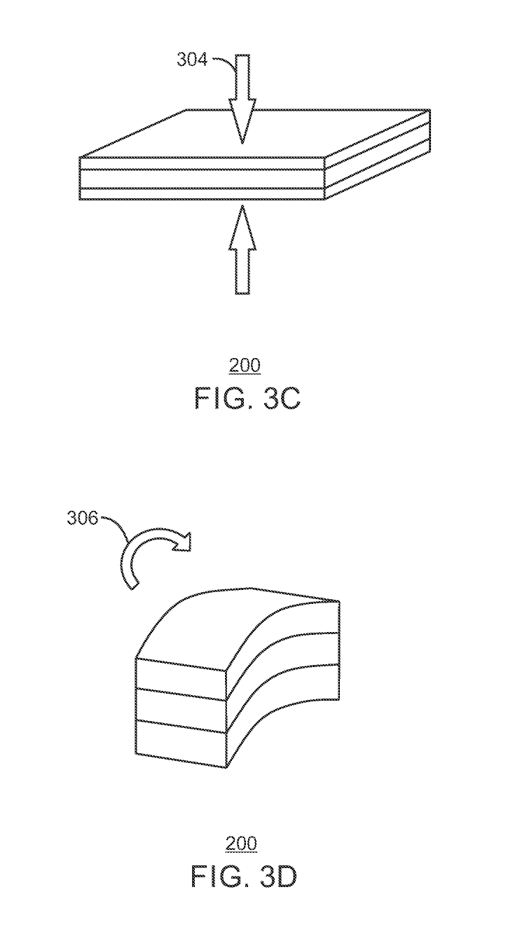

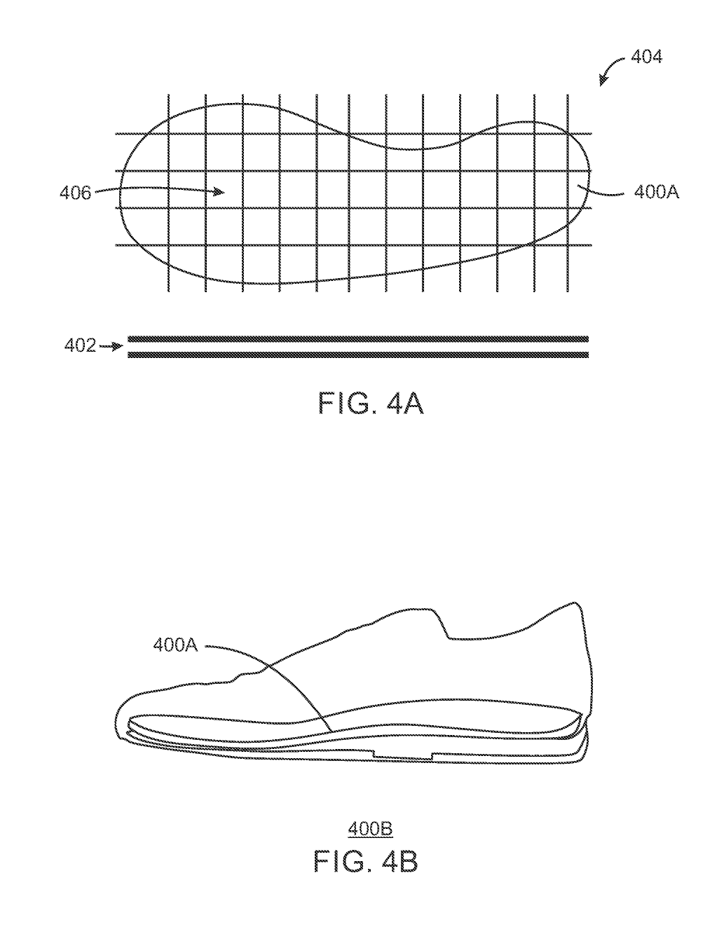

FIG. 4A is an illustration of an electronic skin sole insert 400A. A cross section 402 of the capacitive sensor within the electronic skin sole insert 400A is also illustrated. A grid 404 is illustrated on top of the electronic skin sole insert 400A. In embodiments, the sensor is divided into a plurality of regions be dielectrically separating the regions during manufacture of the sole insert 400A. For example, a section 406 may be dielectrically separated during the printing process of the sole insert 400A for the purpose of focusing on the ball of the foot. Although the electronic skin may be broken into regions according to the particular use of the skin, the sensor of the electronic skin is contiguous, without distinct or separate sensors used to point pressures.

By calculating the grid capacitance profile, a position of the foot can be determined. The electrodes can be stratified such that as a user's foot manipulates sections of the grid, the capacitance of each section is changed. In this way, data from the electronic skin can also be used to determine the forces on the foot in 9 degrees of freedom (tension, compression, torsion on each of three axis) that for example can determine the loading on the foot and through dynamic analysis determine the roll, pitch and yaw associated with the current position of the shoe/person. In embodiments, the roll, pitch and yaw associated with the current position of the shoe is relative to a baseline profile of the shoe. In embodiments, the electronic skin enables a determination of a position or stance of a person. For example, multiple pieces of electronic skin can be applied to various areas of a subject determine the actual position.

In this instantiation as a shoe sensor, a simple flexible strain energy sensor is placed into the insole of the shoe. The sensor is strained (stretched) when a shoe is bent during walking, when the person applies pressure during walking, when the weight transfers from the heel to the ball of the foot, during balancing, during turns and rotating on a pivot foot, or when the person comes to an abrupt stop or accelerates. In each of these cases, strain energy is imparted to the sensor causing a change in shape. In embodiments, strain energy is the effective strain, or total summation of all the strains in the sensor combined as an effective total strain by a mathematical formula. This effective strain can be further analyzed to find a resulting stress and resulting magnitude of load imparted to the sensor network and to the object to which the sensor is applied. Further the effective strain as a result of sensor construction can be decomposed to find the elemental force component directions and magnitudes. Moreover, in examples, shear stress is generated at the sensor in the z plane, changing the capacitance of the sensor. This change in capacitance is interpreted as a load necessary to cause magnitude of capacitance change. The shape change from any stress or change at the sensor causes a capacitance change, which occurs by deformation along the x, y, and z axes and deformation due to rotation. This capacitance change is sent to the wearable device for processing, or storage and post processing. The strain energy may be interpreted as pressure, pressure distribution, force, force vectors, flexure, and shear stress, depending on how the sensor is segmented and arranged.

The electronic skin captures the deformation energy of the user input, in the example of FIG. 4A, from the foot of a user. The electronic skin also enables a sensing capability that is based on the strain energy applied to the sensor allowing capture of a signature based on the deformation of the sensor rather than only pressure. The summation of strain energy and determination of effective force input is used and makes possible the ability to summarize the energy imparted during walking, running, etc. by a user, or the user's unique response to a force imparted by a force external to the user. The sensing and analysis method described herein simplifies multiple complex user inputs to a single resulting force vector capturing the subtleties of the user, enabling a signature to be established and compared. Sensing methods described herein are able to determine deformation and capacitance change from the principal stresses and then combine those stresses to get an effective stress. The effective stress results in a signature, where the signature is a capacitance change with unique inputs. In the example of a shoe insert, the electronic skin can be worn with all shoes and can be implemented as an everyday, all-day monitoring system, with several signatures obtained during the all-day monitoring. Accordingly, the electronic skin can be applied at any location on the user's body as a component of an all-day monitoring system. The electronic skin and strain energy sensing advantages extend to many wearable applications, new purposes, for wearable computing usage models.

FIG. 4B is a cross-section of a shoe 400B with the electronic skin 400A as the sole. In embodiments, and electronic skin 400A including flexible capacitive sensor technology is placed on top the sole of the shoe 400B to provide complex motion analysis. The use of socks with the electronic skin 400A will not affect the performance of the capacitive sensing, as the capacitive sensing of the electronic skin can be calibrated to negate the use of socks. The electronic skin is a flexible capacitor and as the foot applies pressure, deforms the shape (bending), or shear strain from rotational and translation shear occur, the capacitance changes. In embodiments, this capacitance change is the basis for force, pressure, and motion analysis via the computing application analysis. The electronic skin enables the reaction of the foot in all three principle directions (x, y, and z) to be monitored, and rotation can be determined in each direction. This enables complex motion analysis and correlation of the sensor and person to specific activity by the change in capacitance as the user walks, stands, or changes posture during static balancing. Ultimately this leads to a signature of a user in their activities which may be charted, tracked, monitored, or reported. The sole can be changed between multiple shoes, boots, running shoes, cross-trainers, etc., to capture data during an unlimited number of activities.

The electronic skin is not limited to the example of a shoe insert. Any application where activities of a subject can be charted, tracked, monitored, or reported can use an electronic skin. As previously noted, the electronic skin may be applied to the back of a human to continually sense back positioning for detecting of back problems and poor posture. Additionally, the electronic skin may be applied to knees, legs, ankles and thighs to continually track a person's gait, movement of the knee, etc. Similarly, the electronic skin may be applied to the shoulders, arms, and hands to detect the onset of motor skill deficiencies or repetitive motion injuries via tape, adhesives, gloves, wraps, or other articles of clothing that can cover the arms and hands. While particular applications have been used for ease of description, the present techniques are not limited to the uses described. Rather, the electronic skin may be applied in any situation where the pressure and motion from a subject it to be analyzed.

The electronic skin, as an insert of a shoe, can capture data that can track the compression of the foot when walking or standing or balancing. The electronic skin can also capture data that tracks bending when walking and the bend that occurs in the sole of the shoe. The stress values will also capture data relating to axially twisting the electronic skin, such as when the ankle is rotated as this induces shear.

The flexible capacitive sensor is simple, low cost, thin flexible, and can be retrofit to existing sensor applications with a simple drop in design. In this manner, the electronic skin is highly adaptable to multiple applications. Moreover, present techniques enable pressure distribution and motion measurement in “real” shoes—analyzing the actual user environment. Thus, simulations can often result in skewed results as the user is not in their everyday environment. The present techniques avoid simulation.

Capacitive sensing enables data capture at a greater number of points when compared to pressure sensing. Capacitive sensing also results in a continuous collection of data points across the electronic skin. The shoe 400B may also include additional sensors, such as a temperature sensor, accelerometer, or humidity monitoring sensor. In embodiments, the additional sensors are integrated into the electronic skin. The additional sensors may also be sandwiched between layers of the electronic skin. Moreover, a battery component may also be integrated into the electronic skin.

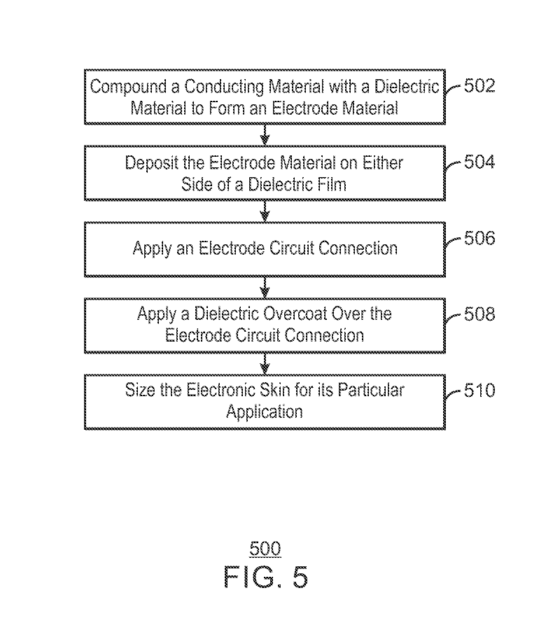

FIG. 5 is a process flow diagram of an example of a method of manufacturing an electronic skin. At block 502, a conducting material can be compounded with a dielectric material to form an electrode material. The conducting material can be any suitable type of conducting material, such as carbon. The dielectric material can be any suitable type of polymer, such as a flexible polymer. For example, the dielectric material can be a silicone material, such as polydimethylsiloxane. The material can be chosen based on the insulation properties of the material and the tactile feel of the material, as well as the elastic modulus of the material, and the ability to compound the dielectric material with a conducting medium.

At block 504, the electrode material can be deposited on either side of a dielectric film. The dielectric film can be any suitable type of polymer. For example, the dielectric film can be a silicone material, such as polydimethylsiloxane. In another example, the dielectric film can be a polyester film, such as a polyethylene terephthalate (PET) film or a biaxially-oriented polyethylene terephthalate (BoPET) film. The electrode material can be deposited on the dielectric film using any suitable deposition method. At block 506, an electrode circuit connection can be applied.

For example, the electrode can be a silicone compounded with a conducting particle. To make the circuit connection, the silicone compounded with the conducting particle can be printed onto the connecting electrode, clamped to the electrode, or coupled to the connecting electrode with any other suitable method.

At block 508, a dielectric overcoat can be applied over the electrode circuit connection. The dielectric overcoat can be any suitable type of insulating material, such as silicone. The dielectric overcoat can be applied by any suitable method, such as printing. This overcoat results in a complete electronic skin. At block 510, the electronic skin is sized for its particular application. For example, the electronic skin may be sized and shaped as an insole of a shoe. The electronic skin may also be formed into a sleeve for an arm or leg, a wrap for a knee, torso, or elbow, and the like.

The process flow diagram of FIG. 5 is not intended to indicate that the method 500 is to include all of the blocks shown in FIG. 5. Further, the method 500 can include any number of additional blocks not shown in FIG. 5, depending on the details of the specific implementation.

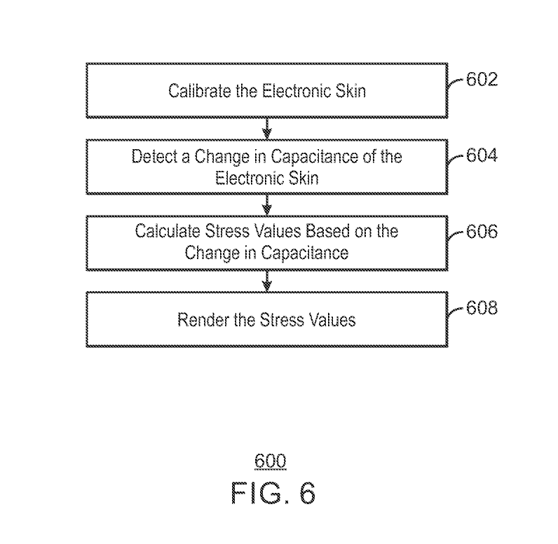

FIG. 6 is a process flow diagram of a method 600 of using an electronic skin. At block 602, the electronic skin is calibrated. In embodiments, the electronic skin is positioned in a baseline position. The deformation of the electronic skin associated with the baseline position is used to establish a baseline profile. The baseline profile may be a baseline pressure profile, and can include baseline shear, tensile, and torsion values. In embodiments, the electronic sensor enables for homogeneous flexural properties (modulus) or intentional differentiation of zone modulus via dielectric separation.

At block 604, a computing device is to detect a capacitance change of the electronic skin. The electronic skin can include a flexible, deformable capacitive sensor. Deformation of the electronic skin can cause a change in capacitance of the electronic skin. The electronic skin can be deformed in a variety of ways, including stretching the electronic skin vertically, stretching the electronic skin horizontally, compressing the electronic skin, bending the electronic skin, twisting the electronic skin, or otherwise deforming the electronic skin. The electronic skin can be deformed by a user's finger or hand.

At block 606, stress values are calculated based on the change in capacitance of the electronic skin. Stress values include shear stress, strain and tensile stress, and torsion of the electronic skin. In embodiments, the stress values are calculated using nine degrees of freedom. This enables accurate capture of complex distortion. In embodiments, electrode resistivity is captured by the electronic skin. The electrode resistivity is used to determine if the capacitance change in primary direction is from tensile or compressive deformation (i.e. the type of force applied to the sensor resulting in determination of motion vector analysis). The electrode resistivity is used to deduce the shape of the deformation that is applied to of the sensor. The deformation may include, but is not limited to stretched, curved, compressed, and direction specific deformations. The computing device can compensates for electrode resistivity drift, and uses dynamic resistivity change in determination of force vectors applied to the electronic skin.

At block 608, the stress values are rendered. The stress values may be rendered on an output device in the form of numerical values or graphics that include charts and plots of the data. The stress values can also be plotted against time. In embodiments, the graphics include videos that replay the movements captured by the electronic skin. The videos may also replay the pressures captured by the electronic skin.

The process flow diagram of FIG. 6 is not intended to indicate that the method 600 is to include all of the blocks shown in FIG. 6. Further, the method 600 can include any number of additional blocks not shown in FIG. 6, depending on the details of the specific implementation.

The present techniques captures data in all directions and along rotational axes. The electronic skin enables deformation detection in multiple directions, resulting in a sensing technology that can be applied to multiple biometric applications.

Example 1 is an electronic skin. The electronic skin includes a flexible capacitive sensor to capture a change in capacitance, wherein the change in capacitance is to translate to sheer, tensile, and torsion values; at least one section, wherein the one section is dielectrically separated from an another section and the sheer, tensile, and torsion values are continuous across the at least one section and the another section.

Example 2 includes the electronic skin of example 1, including or excluding optional features. In this example, the flexible capacitive sensor is calibrated to a normalized operating load.

Example 3 includes the electronic skin of any one of examples 1 to 2, including or excluding optional features. In this example, the flexible capacitive sensor is applied to a subject via tape or an adhesive.

Example 4 includes the electronic skin of any one of examples 1 to 3, including or excluding optional features. In this example, the flexible capacitive sensor comprises an insulator.

Example 5 includes the electronic skin of any one of examples 1 to 4, including or excluding optional features. In this example, the shear, tensile and torsion values correlate to a force applied to deform the flexible capacitive sensor.

Example 6 includes the electronic skin of any one of examples 1 to 5, including or excluding optional features. In this example, the electronic skin comprises at least two electrodes and a dielectric between the electrodes.

Example 7 includes the electronic skin of any one of examples 1 to 6, including or excluding optional features. In this example, the electronic skin comprises a battery.

Example 8 includes the electronic skin of any one of examples 1 to 7, including or excluding optional features. In this example, the flexible capacitive sensor comprises at least two electrodes and a dielectric between the electrodes, and wherein the electrodes comprise a silicone compounded with a conducting medium.

Example 9 includes the electronic skin of any one of examples 1 to 8, including or excluding optional features. In this example, the flexible capacitive sensor is deformed by compressing the flexible capacitive sensor, stretching the flexible capacitive sensor vertically, stretching the flexible capacitive sensor horizontally, bending the flexible capacitive sensor, twisting the flexible capacitive sensor, or any combination thereof.

Example 10 includes the electronic skin of any one of examples 1 to 9, including or excluding optional features. In this example, strain components from nine degrees of freedom are combined to determine effective a strain and equivalent force magnitude.

Example 11 includes the electronic skin of any one of examples 1 to 10, including or excluding optional features. In this example, a resultant force vector observed at the electronic skin is decomposed into elemental force components and directions.

Example 12 includes the electronic skin of any one of examples 1 to 11, including or excluding optional features. In this example, the change in capacitance is decomposed into component capacitance change, and resulting force components are determined. Optionally, the electronic skin includes cataloging the resulting force components simultaneously.

Example 13 includes the electronic skin of any one of examples 1 to 12, including or excluding optional features. In this example, the electronic skin is a shoe insert.

Example 14 includes the electronic skin of any one of examples 1 to 13, including or excluding optional features. In this example, the electronic skin is a sleeve.

Example 15 includes the electronic skin of any one of examples 1 to 14, including or excluding optional features. In this example, the electronic skin is to be applied as a wrap.

Example 16 includes the electronic skin of any one of examples 1 to 15, including or excluding optional features. In this example, the flexible capacitive sensor comprises a flexible polymer.

Example 17 is a system with an electronic skin. The system includes an electronic skin, wherein the electronic skin is a flexible capacitive sensor that is to capture a change in capacitance; a processor communicatively coupled to the electronic skin, wherein when the processor is to execute instructions, the processor is to translate the change in capacitance to pressure and motion values; and a display to render the pressure and motion values.

Example 18 includes the system of example 17, including or excluding optional features. In this example, the flexible capacitive sensor is calibrated to a normalized operating load.

Example 19 includes the system of any one of examples 17 to 18, including or excluding optional features. In this example, the electronic skin comprises at least one section, wherein the one section is dielectrically separated from another section and the sheer, tensile, and torsion values are continuous across the at least one section and the another section.

Example 20 includes the system of any one of examples 17 to 19, including or excluding optional features. In this example, the pressure and motion values comprise sheer, tensile, and torsion values.

Example 21 includes the system of any one of examples 17 to 20, including or excluding optional features. In this example, the electronic skin comprises at least two electrodes and a dielectric between the electrodes.

Example 22 includes the system of any one of examples 17 to 21, including or excluding optional features. In this example, the system comprises a battery.

Example 23 includes the system of any one of examples 17 to 22, including or excluding optional features. In this example, the electronic skin is a shoe insert. Optionally, the change of the capacitance is to initiate calculations at a computing device. Optionally, the calculations are to correlate to a force applied to deform the flexible capacitive sensor.

Example 24 includes the system of any one of examples 17 to 23, including or excluding optional features. In this example, the electronic skin is a sleeve. Optionally, the change of the capacitance is to initiate calculations at a computing device. Optionally, the calculations are to correlate to a force applied to deform the flexible capacitive sensor.

Example 25 includes the system of any one of examples 17 to 24, including or excluding optional features. In this example, the electronic skin is to be applied as a wrap. Optionally, the change of the capacitance is to initiate calculations at a computing device. Optionally, the calculations are to correlate to a force applied to deform the flexible capacitive sensor.

Example 26 is a computing device. The computing device includes a processor, a memory, and logic stored in the memory to be executed by the processor, comprising: logic to calibrate an electronic skin; logic to detect a change in capacitance of the electronic skin; logic to calculate stress values based on the change in capacitance; and logic to render the stress values.

Example 27 includes the computing device of example 26, including or excluding optional features. In this example, the electronic skin is calibrated in a baseline position.

Example 28 includes the computing device of any one of examples 26 to 27, including or excluding optional features. In this example, the change in capacitance of the electronic skin is caused by a deformation of the electronic skin.

Example 29 includes the computing device of any one of examples 26 to 28, including or excluding optional features. In this example, stress values comprise shear stress, strain and tensile stress, and torsion of the electronic skin.

Example 30 includes the computing device of any one of examples 26 to 29, including or excluding optional features. In this example, the stress values are calculated using nine degrees of freedom.

Example 31 includes the computing device of any one of examples 26 to 30, including or excluding optional features. In this example, a flexible capacitive sensor of the electronic skin comprises at least two electrodes and a dielectric between the electrodes. Optionally, the flexible capacitive sensor comprises a flexible polymer. Optionally, the flexible capacitive sensor comprises at least two electrodes and a dielectric between the electrodes, and wherein the electrodes comprise a silicone compounded with a conducting medium. Optionally, the flexible capacitive sensor is deformed by compressing the flexible capacitive sensor, stretching the flexible capacitive sensor vertically, stretching the flexible capacitive sensor horizontally, bending the flexible capacitive sensor, twisting the flexible capacitive sensor, or any combination thereof. Optionally, a thickness of the flexible capacitive sensor is less than 500 μm. Optionally, the flexible capacitive sensor comprises a sensing range of 5 grams to 5 kg. Optionally, the flexible capacitive sensor comprises a supportable strain of at least 350%.

Example 32 is a tangible, non-transitory, computer-readable medium. The computer-readable medium includes instructions that direct the processor to calibrate an electronic skin; detect a change in capacitance of the electronic skin; calculate stress values based on the change in capacitance; and render the stress values.

Example 33 includes the computer-readable medium of example 32, including or excluding optional features. In this example, the electronic skin is calibrated in a baseline position.

Example 34 includes the computer-readable medium of any one of examples 32 to 33, including or excluding optional features. In this example, the change in capacitance of the electronic skin is caused by a deformation of the electronic skin.

Example 35 includes the computer-readable medium of any one of examples 32 to 34, including or excluding optional features. In this example, stress values comprise shear stress, strain and tensile stress, and torsion of the electronic skin.

Example 36 includes the computer-readable medium of any one of examples 32 to 35, including or excluding optional features. In this example, the stress values are calculated using nine degrees of freedom.

Example 37 includes the computer-readable medium of any one of examples 32 to 36, including or excluding optional features. In this example, a flexible capacitive sensor of the electronic skin comprises at least two electrodes and a dielectric between the electrodes. Optionally, the flexible capacitive sensor comprises a flexible polymer. Optionally, the flexible capacitive sensor comprises at least two electrodes and a dielectric between the electrodes, and wherein the electrodes comprise a silicone compounded with a conducting medium. Optionally, the flexible capacitive sensor is deformed by compressing the flexible capacitive sensor, stretching the flexible capacitive sensor vertically, stretching the flexible capacitive sensor horizontally, bending the flexible capacitive sensor, twisting the flexible capacitive sensor, or any combination thereof. Optionally, a thickness of the flexible capacitive sensor is less than 500 μm. Optionally, the flexible capacitive sensor comprises a sensing range of 5 grams to 5 kg. Optionally, the flexible capacitive sensor comprises a supportable strain of at least 350%.

Example 38 is an apparatus for capacitive sensing. The apparatus includes instructions that direct the processor to a means to capture a change in capacitance, wherein the change in capacitance is to translate to sheer, tensile, and torsion values; at least one section, wherein the one section is dielectrically separated from another section and the sheer, tensile, and torsion values are continuous across the at least one section and the another section.

Example 39 includes the apparatus of example 38, including or excluding optional features. In this example, the means to capture the change in capacitance is calibrated to a normalized operating load.

Example 40 includes the apparatus of any one of examples 38 to 39, including or excluding optional features. In this example, the means to capture the change in capacitance is applied to a subject via tape or an adhesive.

Example 41 includes the apparatus of any one of examples 38 to 40, including or excluding optional features. In this example, the means to capture the change in capacitance comprises an insulator.

Example 42 includes the apparatus of any one of examples 38 to 41, including or excluding optional features. In this example, the shear, tensile and torsion values correlate to a force applied to deform the means to capture the change in capacitance.

Example 43 includes the apparatus of any one of examples 38 to 42, including or excluding optional features. In this example, the means to capture the change in capacitance comprises at least two electrodes and a dielectric between the electrodes.

Example 44 includes the apparatus of any one of examples 38 to 43, including or excluding optional features. In this example, the means to capture the change in capacitance comprises a battery.

Example 45 includes the apparatus of any one of examples 38 to 44, including or excluding optional features. In this example, the means to capture the change in capacitance comprises at least two electrodes and a dielectric between the electrodes, and wherein the electrodes comprise a silicone compounded with a conducting medium.

Example 46 includes the apparatus of any one of examples 38 to 45, including or excluding optional features. In this example, the means to capture the change in capacitance is deformed by compressing the means to capture the change in capacitance, stretching the means to capture the change in capacitance vertically, stretching the means to capture the change in capacitance horizontally, bending the means to capture the change in capacitance, twisting the means to capture the change in capacitance, or any combination thereof.

Example 47 includes the apparatus of any one of examples 38 to 46, including or excluding optional features. In this example, the means to capture the change in capacitance is a shoe insert.

Example 48 includes the apparatus of any one of examples 38 to 47, including or excluding optional features. In this example, the means to capture the change in capacitance is a sleeve.

Example 49 includes the apparatus of any one of examples 38 to 48, including or excluding optional features. In this example, the means to capture the change in capacitance is to be applied as a wrap.

In the foregoing description and claims, the terms “coupled” and “connected,” along with their derivatives, can be used. It should be understood that these terms are not intended as synonyms for each other. Rather, in particular embodiments, “connected” can be used to indicate that two or more elements are in direct physical or electrical contact with each other. “Coupled” can mean that two or more elements are in direct physical or electrical contact. However, “coupled” can also mean that two or more elements are not in direct contact with each other, but yet still co-operate or interact with each other.

Some embodiments can be implemented in one or a combination of hardware, firmware, and software. Some embodiments can also be implemented as instructions stored on a machine-readable medium, which can be read and executed by a computing platform to perform the operations described herein. A machine-readable medium can include any mechanism for storing or transmitting information in a form readable by a machine, e.g., a computer. For example, a machine-readable medium can include read only memory (ROM); random access memory (RAM); magnetic disk storage media; optical storage media; flash memory devices; or electrical, optical, acoustical or other form of propagated signals, e.g., carrier waves, infrared signals, digital signals, or the interfaces that transmit and/or receive signals, among others.

An embodiment is an implementation or example. Reference in the specification to “an embodiment,” “one embodiment,” “some embodiments,” “various embodiments,” or “other embodiments” means that a particular feature, structure, or characteristic described in connection with the embodiments is included in at least some embodiments, but not necessarily all embodiments, of the inventions. The various appearances of “an embodiment,” “one embodiment,” or “some embodiments” are not necessarily all referring to the same embodiments. Elements or aspects from an embodiment can be combined with elements or aspects of another embodiment.

Not all components, features, structures, characteristics, etc. described and illustrated herein need be included in a particular embodiment or embodiments. If the specification states a component, feature, structure, or characteristic “can”, “might”, “can” or “could” be included, for example, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

It is to be noted that, although some embodiments have been described in reference to particular implementations, other implementations are possible according to some embodiments. Additionally, the arrangement and/or order of circuit elements or other features illustrated in the drawings and/or described herein need not be arranged in the particular way illustrated and described. Many other arrangements are possible according to some embodiments.

In each system shown in a figure, the elements in some cases can each have a same reference number or a different reference number to suggest that the elements represented could be different and/or similar. However, an element can be flexible enough to have different implementations and work with some or all of the systems shown or described herein. The various elements shown in the figures can be the same or different. Which one is referred to as a first element and which is called a second element is arbitrary.

In the preceding description, various aspects of the disclosed subject matter have been described. For purposes of explanation, specific numbers, systems and configurations were set forth in order to provide a thorough understanding of the subject matter. However, it is apparent to one skilled in the art having the benefit of this disclosure that the subject matter can be practiced without the specific details. In other instances, well-known features, components, or modules were omitted, simplified, combined, or split in order not to obscure the disclosed subject matter.

While the disclosed subject matter has been described with reference to illustrative embodiments, this description is not intended to be construed in a limiting sense. Various modifications of the illustrative embodiments, as well as other embodiments of the subject matter, which are apparent to persons skilled in the art to which the disclosed subject matter pertains are deemed to lie within the scope of the disclosed subject matter.

While the present techniques can be susceptible to various modifications and alternative forms, the exemplary examples discussed above have been shown only by way of example. It is to be understood that the technique is not intended to be limited to the particular examples disclosed herein. Indeed, the present techniques include all alternatives, modifications, and equivalents falling within the true spirit and scope of the appended claims.

What is claimed is:

1. An electronic skin, comprising:
a flexible capacitive sensor to capture a change in capacitance, wherein the change in capacitance is to translate to sheer, tensile, and torsion values;
at least one section, wherein the one section is dielectrically separated from an another section and the sheer, tensile, and torsion values are continuous across the at least one section and the another section.

2. The electronic skin of claim 1, wherein the flexible capacitive sensor is calibrated to a normalized operating load.

3. The electronic skin of claim 1, wherein the flexible capacitive sensor is applied to a subject via tape or an adhesive.

4. The electronic skin of claim 1, wherein the flexible capacitive sensor comprises an insulator.

5. The electronic skin of claim 1, wherein the shear, tensile and torsion values correlate to a force applied to deform the flexible capacitive sensor.

6. The electronic skin of claim 1, wherein the electric skin comprises at least two electrodes and a dielectric between the electrodes.

7. The electronic skin of claim 1, wherein the electric skin includes a battery.

8. The electronic skin of claim 1, wherein the flexible sensor comprises at least two electrodes and a dielectric between the electrodes, and wherein the electrodes comprise a silicone compounded with a conducting medium.

9. The electronic skin of claim 1, wherein the flexible sensor is deformed by compressing the flexible sensor, stretching the flexible sensor vertically, stretching the flexible sensor horizontally, bending the flexible sensor, twisting the flexible sensor, or any combination thereof.

10. The electronic skin of claim 1, wherein strain components from nine degrees of freedom are combined to determine effective a strain and equivalent force magnitude.

11. The electronic skin of claim 1, wherein a resultant force vector observed at the electronic skin is decomposed into elemental force components and directions.

12. The electronic skin of claim 1, wherein the change in capacitance is decomposed into component capacitance change, and resulting force components are determined.

13. The electronic skin of claim 12, comprising cataloging the resulting force components simultaneously.

14. A system with an electronic skin, comprising:
an electronic skin, wherein the electronic skin is a flexible capacitive sensor that is to capture a change in capacitance;
a processor communicatively coupled to the electronic skin, wherein when the processor is to execute instructions, the processor is to translate the change in capacitance to pressure and motion values;
a display to render the pressure and motion values.

15. The system of claim 14, wherein the flexible capacitive sensor is calibrated to a normalized operating load.

16. The system of claim 14, wherein the electric skin comprises at least one section, wherein the one section is dielectrically separated from another section and the sheer, tensile, and torsion values are continuous across the at least one section and the another section.

17. The system of claim 14, wherein the pressure and motion values include sheer, tensile, compressive, and torsion values.

18. The system of claim 14, wherein the electric skin comprises at least two electrodes and a dielectric between the electrodes.

19. The system of claim 14, wherein the electric skin is a shoe insert.

20. The system of claim 14, wherein the electric skin is a sleeve.

21. A computing device, comprising:
a processor, a memory, and logic stored in the memory to be executed by the processor, comprising:
logic to calibrate an electronic skin;
logic to detect a change in capacitance of the electronic skin;
logic to calculate force and direction values that induced the change in capacitance; and
logic to render the force and direction values.

22. The computing device of claim 21, wherein the electronic skin is calibrated in a baseline position.

23. The computing device of claim 21, wherein the change in capacitance of the electronic skin is caused by a deformation of the electronic skin.

24. The computing device of claim 21, wherein stress values include shear, strain, tensile, compressive, and torsion stress of the electronic skin.

25. The computing device of claim 21, wherein the stress values are calculated using nine degrees of freedom.
